(19) United States
(12) Patent Application Publication
(10) Pub. No.: US 2025/0221813 A1
(43) Pub. Date: Jul. 10, 2025

(54) Negative Poissons Ratio Materials for Artificial Hair

(71) Applicant: Joon Bu Park, Las Vegas, NV (US)

(72) Inventor: Joon Bu Park, Las Vegas, NV (US)

(21) Appl. No.: 18/408,735

(22) Filed: Jan. 10, 2024

(51) Int. Cl. A61F 2/10; A61F 2/00

(52) U.S. Cl. CPC A61F 2/10; A61F 2002/0081; A61F 2210/0057; A61F 2210/0076; A61F 2250/0039

(57) ABSTRACT

An artificial hair includes an elongated artificial hair element having a negative Poisson's ratio (NPR) material with a Poisson's ratio of between 0 and −1. The artificial hair element can be implanted into human tissue. A method for making an artificial hair includes forming an elongated artificial hair element from a negative Poisson's ratio (NPR) material having a Poisson's ratio of between 0 and −1.

### BACKGROUND

The present disclosure relates generally to materials for artificial hair.

Artificial hair integration or hair extensions can be utilized for various purposes, such as to add length and fullness to human hair. Medical procedures, such as follicular transplants and follicular strip harvesting, can be employed for hair transplantation. Other hair enhancement techniques can include hairpieces and wigs made from artificial hair fibers.

### SUMMARY

We describe here artificial hair fibers for hair enhancement and hair transplants that include materials having a negative Poisson's ratio (“NPR materials”). Throughout this specification, “artificial hair fibers” can refer to hair fibers that are made from synthetic (e.g., non-naturally occurring) materials, and natural hair fibers (e.g., human or animal hair fibers) that have been synthetically-modified, e.g., modified using synthetic materials or subjected to any other appropriate structural modification. Generally, the artificial hair fibers described in this specification can be used for a variety of purposes. In some cases, the artificial hair fibers can be implanted into human tissue for hair restoration and/or replacement. In some cases, the artificial hair fibers can be used for enhancement purposes, e.g., as part of hair extensions, wigs, weaves, or any other appropriate hair enhancement article. In some cases, the artificial hair fibers described in this specification can be used for making various types of objects, e.g., clothing articles, or any other appropriate types of objects.

Artificial hair fibers that include NPR materials can exhibit a variety of physical properties that can make them superior to typical artificial hair fibers that do not include such materials. For example, artificial hair fibers that include NPR materials can expand in transverse direction in response to expansion in longitudinal direction, e.g., when stretched. This can promote hair fullness and significantly increase the longevity of artificial hair fibers when they are implanted into human tissue or worn as a hair piece. Furthermore, artificial hair fibers that include NPR materials can be substantially lightweight, and can exhibit superior mechanical strength and thermal resistance, when compared to conventional artificial hair fibers, thereby effectively adding length and volume to natural human hair, and reducing wear and strain in hair implants and hair pieces over time.

According to a first aspect, there is provided an artificial hair that includes: an elongated artificial hair element comprising a negative Poisson's ratio (NPR) material having a Poisson's ratio of between 0 and −1.

In some implementations, the elongated artificial hair element is configured to be implanted into human tissue.

In some implementations, the elongated artificial hair element further comprises a positive Poisson's ratio (PPR) material.

In some implementations, the artificial hair of claim 1, further includes: a coating covering at least a portion of an outer surface of the elongated artificial hair element, where the coating includes the negative Poisson's ratio (NPR) material.

In some implementations, the coating includes one or more elongated fibers of the negative Poisson's ratio (NPR) material that at least partially extend through the elongated artificial hair element.

In some implementations, the coating includes multiple particles of the negative Poisson's ratio (NPR) material.

In some implementations, the coating includes a matrix of the negative Poisson's ratio (NPR) material and a positive Poisson's ratio (PPR) material.

In some implementations, the elongated artificial hair element includes a body having an increasing diameter from a first end to a second end, the second end comprising a root portion that is configured to be implanted into human tissue.

In some implementations, the root portion includes the negative Poisson's ratio (NPR) material having a Poisson's ratio of between 0 and −1.

In some implementations, a portion of the elongated artificial hair element other than the root portion includes a positive Poisson's ratio (PPR) material.

In some implementations, the root portion includes: one or more elongated fibers of the negative Poisson's ratio (NPR) material that at least partially extend through the root portion, or a plurality of particles of the negative Poisson's ratio (NPR) material.

In some implementations, the negative Poisson's ratio (NPR) material is a porous negative Poisson's ratio (NPR) material.

In some implementations, the root portion includes a matrix of the negative Poisson's ratio (NPR) material and a positive Poisson's ratio (PPR) material.

In some implementations, the elongated artificial hair element includes a core portion that extends through the elongated artificial hair element, and wherein the core portion includes the negative Poisson's ratio (NPR) material.

In some implementations, the core portion includes one or more elongated fibers of the negative Poisson's ratio (NPR) material that at least partially extend through the elongated artificial hair element.

In some implementations, the core portion includes a plurality of particles of the negative Poisson's ratio (NPR) material.

In some implementations, the core portion includes a matrix of the negative Poisson's ratio (NPR) material and a positive Poisson's ratio (PPR) material.

According to a second aspect, there is provided a method for making an artificial hair, the method including: forming an elongated artificial hair element from a negative Poisson's ratio (NPR) material having a Poisson's ratio of between 0 and −1.

In some implementations, forming the elongated artificial hair element includes forming the elongated artificial hair element using an additive manufacturing technique.

In some implementations, forming the elongated artificial hair element includes: forming the elongated artificial hair element from a matrix of the negative Poisson's ratio (NPR) material and a positive Poisson's ratio material (PPR).

The details of one or more embodiments of the subject matter of this specification are set forth in the accompanying drawings and the description below. Other features, aspects, and advantages of the subject matter will become apparent from the description, the drawings, and the claims.

### DESCRIPTION OF DRAWINGS

FIG. 1 illustrates an example natural hair fiber.

FIG. 2A illustrates an example structure of a natural hair fiber.

FIG. 2B illustrates an example outer layer of a natural hair fiber.

FIG. 3 illustrates example materials with negative and positive Poisson's ratios.

FIG. 4 illustrates an example change in diameter of materials with negative and positive Poisson's ratios.

FIG. 5 illustrates an example cross-sectional view of artificial hair fibers made from materials with negative and positive Poisson's ratios.

FIG. 6 illustrates an example composite material having both negative and positive Poisson's ratios.

FIG. 7A illustrates an example artificial hair fiber made from a composite material having both negative and positive Poisson's ratios.

FIG. 7B illustrates an example artificial hair fiber partially made from materials with negative Poisson's ratios.

Like reference numbers and designations in the various drawings indicate like elements.

### DETAILED DESCRIPTION

We describe here artificial hair fibers that are at least partially formed of materials having a negative Poisson's ratio (“NPR materials,” also referred to as auxetic materials). As described in more detail below, such artificial hair fibers can be used with various types of hair enhancement techniques. An inner portion (e.g., a core), an outer portion (e.g., a coating), and a root portion, of an artificial hair fiber, or a combination thereof, can be at least partially formed from an NPR material. This composition can facilitate reduced weight, increased thermal resistance, increased mechanical strength, increased hair fullness, and improved stress and strain characteristics of artificial hair fibers over time, thereby effectively adding length and volume to human hair.

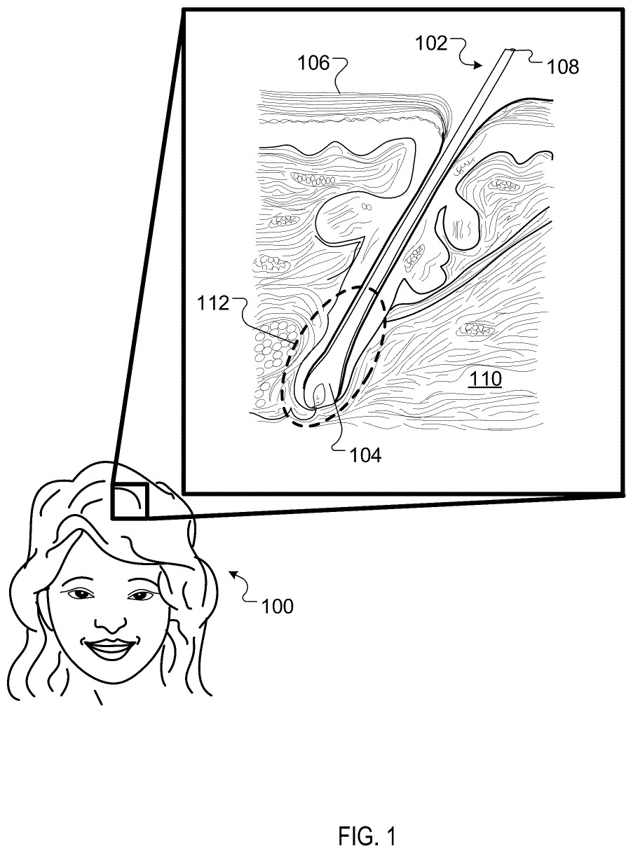

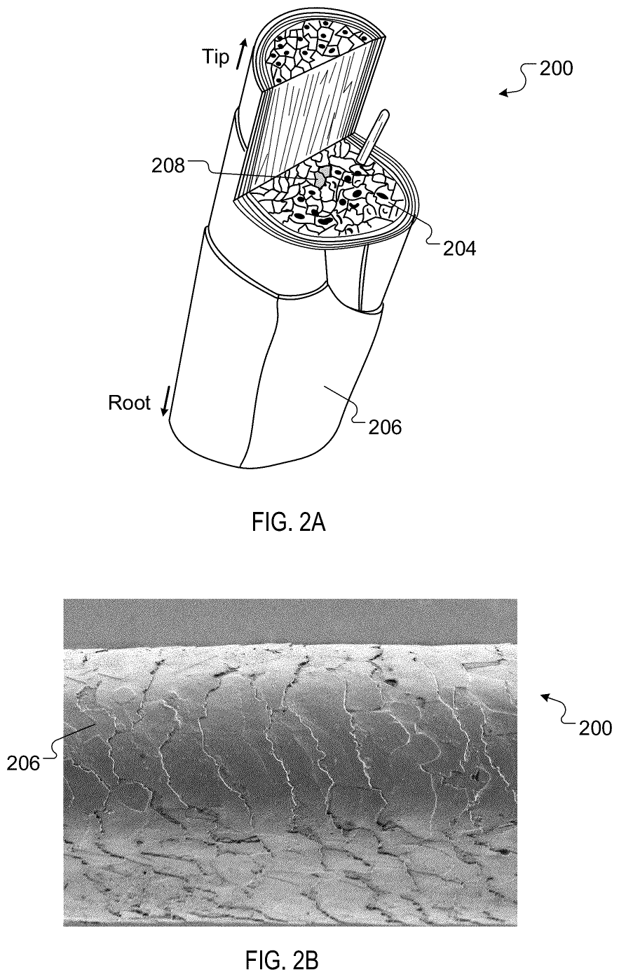

FIG. 1 illustrates an example natural hair fiber 102 of natural human hair 100. Generally, natural hair fiber 102 visibly appears as protruding from a deeper layer 110 (e.g., the dermis) under a surface 106 of the skin. In particular, the natural hair fiber 102 extends from a hair follicle 112 in the dermis 110 that includes a hair root 106, e.g., a root portion of the hair fiber 102. The natural hair fiber 102 further includes a hair shaft 108 that is a portion of the natural hair fiber 102 that extends above the skin surface 106. FIG. 2A illustrates an example structure of a natural hair fiber 200 (e.g., the natural hair fiber 102 in FIG. 1) in more detail. The natural hair fiber 200 includes an inner medulla 208, a cortex 204, and an outer cuticle 206. The cuticle 206 is a transparent outer layer (e.g., a coating) of the natural hair fiber 102. As illustrated in FIG. 2B, the cuticle 206 is made of scales that generally overlap one another and protect inner layers of the natural hair fiber 102. The medulla 208 is the innermost layer (e.g., a core portion) of the hair fiber 200.

As described in more detail below, the structure of an artificial hair fiber can similarly include an elongated artificial hair element having an innermost (e.g., core) portion, an outer (e.g., coating) portion, and a root portion. Typical artificial hair fibers are made from materials such as, e.g., acrylic, modacrylic, nylon, polyester, polymers, or any other appropriate positive Poisson's ratio material (also referred to as a “PPR material”). There exist various types of hair enhancement techniques that utilize artificial hair fibers either in addition to, or instead of, natural hair fibers 200, in order to improve an appearance of human hair.

The hair enhancement techniques can be broadly divided into surgical and non-surgical modalities. The objective of hair enhancement techniques typically involves adding artificial hair fibers to the body in order to achieve a high quality imitation of naturally-looking hair. The non-surgical modalities can generally include the wearing and integration of different types of head pieces, such as wigs, weaves, extensions, and other types of hair pieces, made from artificial hair fibers. However, such non-surgical modalities of artificial hair integration using typical artificial hair fibers pose numerous challenges and constraints. For example, typical artificial hair fibers often have only a limited heat resistance which prevents the wearer of the hair piece from using heat appliances for styling purposes. Furthermore, typical artificial hair fibers often lack flexibility and are prone to static buildup causing frizz and unmanageable tangling. Moreover, typical artificial hair fibers are subject to significant wear and tear with an average lifespan of only a few months. In addition, artificial hair fiber materials often look unnatural and do not match hair density patterns of natural human hair. Other types of hair enhancement techniques can include hair transplantation, which is a surgical technique that removes a natural hair follicle (e.g., the hair follicle 112 in FIG. 1) from a donor site of the body to a recipient side of the body, e.g., a bald or balding part. However, such surgical hair restoration techniques often fail to provide a natural-looking texture, density, and fullness of hair, and may result in further hair thinning and hair loss in the future.

With hair enhancement techniques such as those described above, involving either integration of artificial hair fibers or implantation of natural hair fibers, the resulting appearance can heavily rely on the quality of hair fiber material. As described in more detail below, hair enhancement techniques can be greatly improved by utilizing artificial hair fibers that are at least partially formed of materials having a negative Poisson's ratio (“NPR materials”).

An NPR material is a material that has a Poisson's ratio that is less than zero, such that when the material experiences a positive strain along one axis (e.g., when the material is stretched), the strain in the material along the two perpendicular axes is also positive (e.g., the material expands in cross-section). Conversely, when the material experiences a negative strain along one axis (e.g., when the material is compressed), the strain in the material along a perpendicular axis is also negative (e.g., the material compresses along the perpendicular axis). By contrast, a material with a positive Poisson's ratio (a “PPR material”) has a Poisson's ratio that is greater than zero. When a PPR material experiences a positive strain along one axis (e.g., when the material is stretched), the strain in the material along the two perpendicular axes is negative (e.g., the material compresses in cross-section), and vice versa.

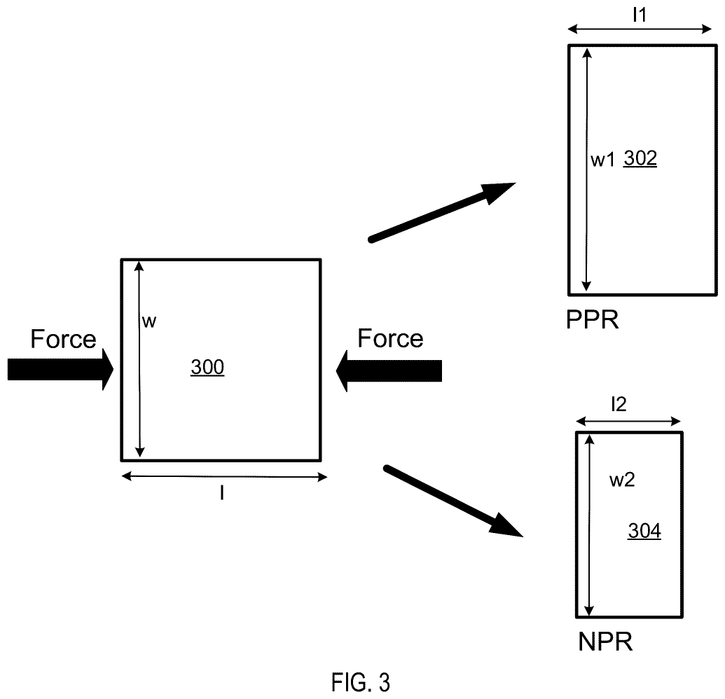

Materials with negative and positive Poisson's ratios are illustrated in FIG. 3, which depicts a hypothetical two-dimensional block of material 300 with length l and width w.

If the hypothetical block of material 300 is a PPR material, when the block of material 300 is compressed along its width w, the material deforms into the shape shown as block 302. The width w1 of block 302 is less than the width w of block 300, and the length l1 of block 302 is greater than the length l of block 300: the material compresses along its width and expands along its length.

By contrast, if the hypothetical block of material 300 is an NPR material, when the block of material 300 is compressed along its width w, the material deforms into the shape shown as block 304. Both the width w2 and the length l2 of block 304 are less than the width w and length l, respectively, of block 300: the material compresses along both its width and its length. These properties of NPR materials make them particularly suitable for integration into artificial hair fibers. For example, because NPR materials expand in transverse direction in response to expansion in longitudinal direction, e.g., when stretched, this can promote fullness and mechanical strength of artificial hair fibers that are at least partially formed from such materials. As a particular example, when artificial hair fibers that include NPR materials are implanted into human tissue, e.g., through a surgical hair transplantation technique, pulling on the artificial hair fibers can cause them to expand in transverse direction, thereby increasing the resistance of artificial hair fibers from being torn out.

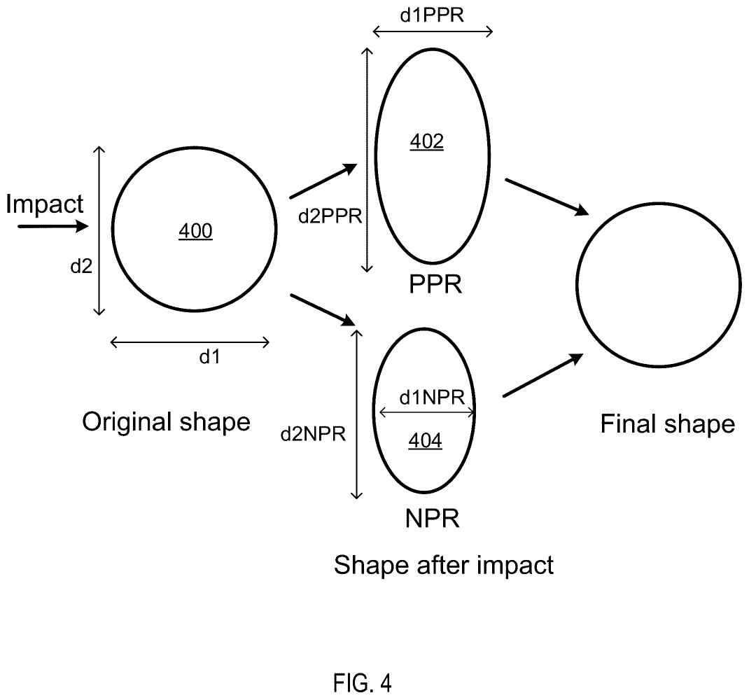

FIG. 4 illustrates an example change in diameter of a material 400 upon impact. Although the material 400 in FIG. 4 is shown as a rounded ball, a similar deformation occurs in materials of other shapes. Prior to impact, the material 400 has a diameter d1 in the direction of the impact and a diameter d2 in the direction perpendicular to the impact. If the material 400 is a PPR material, the material undergoes significant deformation (e.g., elastic deformation) into a shape 402, such that the diameter in the direction of the impact decreases to d1PPR and the diameter in the direction perpendicular to the impact increases to d2PPR. By contrast, if the material 400 is an NPR material, the material undergoes less extensive deformation into a shape 404. The diameter of the shape 404 in the direction of the impact decreases to d1NPR, which is approximately the same as d1PPR. However, the diameter of the shape 404 in the direction perpendicular to the impact also decreases, to d2NPR. The magnitude of the difference between d2 and d2NPR is less than the magnitude of the difference between d2 and d2PPR, meaning that the NPR material undergoes less deformation than the PPR material. Accordingly, an artificial hair fiber that includes the NPR material can effectively concentrate itself under the compressive load upon impact, thereby allowing the artificial hair fiber to better resist the load. In some cases, the artificial hair fiber that includes the NPR material can become stiffer and stronger when the amplitude of the load increases. These properties of the NPR material can significantly improve strain and stress characteristics of artificial hair fibers that include such materials, thereby improving density, longevity, and fullness of artificial hair.

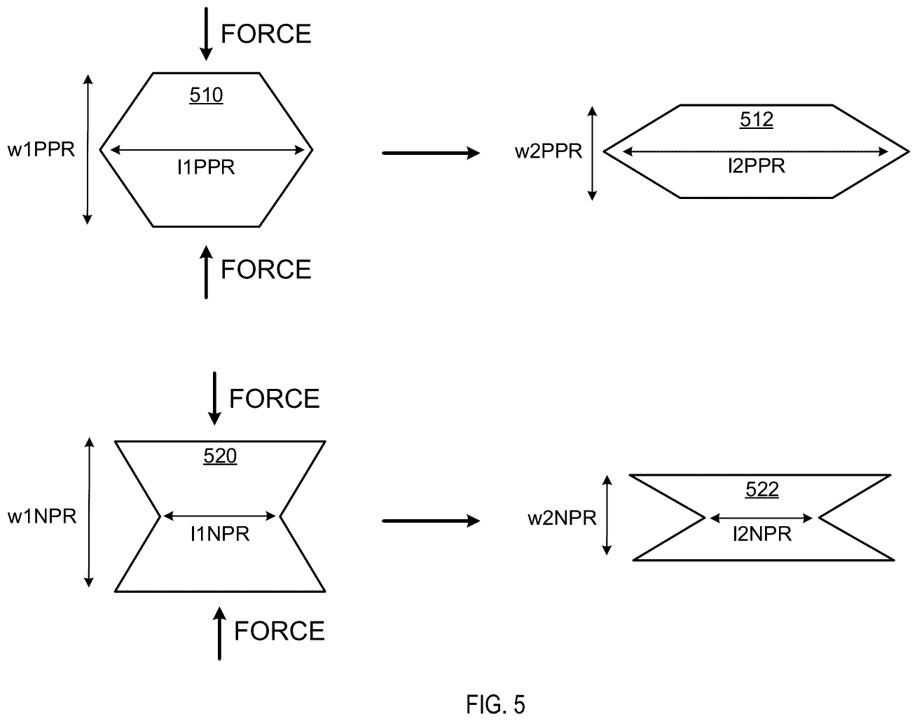

FIG. 5 illustrates an example cross-sectional view of artificial hair fibers made from materials with negative and positive Poisson's ratios. Generally, artificial hair fibers can have a cross-section having any appropriate shape, e.g., circular shape, elliptical shape, or any other appropriate shape. The cross-section of the artificial hair fiber 510 made from a material that exhibits a positive Poisson's ratio (PPR) has length l1PPR and width w1PPR, with sides that are angled outwards away from a central region of the fiber 510, defining a convex profile. When compression (e.g., “FORCE”) is applied to opposing walls of the cross-section of the fiber 510, the fiber 510 deforms into a shape as shown by a deformed fiber 512. The width w2PPR of the cross-section of the fiber 512 is less than the width w1PPR of the cross-section of the fiber 510, and the length l2PPR of the cross-section of the fiber 512 is greater than the length l1PPR of the cross-section of the fiber 510: the material compresses along its width and expands along its length such that the cross-section of the fiber 510 exhibits a positive Poisson's ratio. Specifically, the angled region of each side of the cross-section of the fiber 510 collapses outwards, increasing the lateral dimension of the cross-section.

The cross-section of the artificial hair fiber 520 made from a material that exhibits a negative Poisson's ratio (NPR) has width w1NPR, with two sides that are concave, e.g., protrude inwards towards the interior of the cross-section. The internal length between two concave sides of the cross-section is l1NPR. When compression (e.g., “FORCE) is applied to flat sides of the cross-section of the artificial hair fiber 520, it causes the two other inwardly-directed sides to buckle inward further, causing the cross-section of the artificial hair fiber 520 to compress. In other words, the cross-section of the artificial hair fiber 520 compresses in all directions, as illustrated by a deformed shape 522 of the artificial hair fiber 522. Specifically, the width w2NPR of the cross-section of the fiber 522 is less than the width w1NPR of the cross-section of the fiber 520, and the length l2NPR of the cross-section of the fiber 522 is also less than the length l1NPR of the cross-section of the fiber 520: the cross-section of the artificial hair fiber 520 compresses along both its width and its length such that the cross-section exhibits a negative Poisson's ratio.

Similarly, tension applied to opposing sides of the cross-section of the artificial hair fiber 520 will cause the two inwardly directed sides of the cross-section to unfold, causing the cross-section to expand, such that expansion occurs in all directions. NPR materials can generally have a Poisson's ratio of between −1 and 0, e.g., between −0.8 and 0, e.g., −0.8, −0.7, −0.6, −0.5, −0.4, −0.3, −0.2, or −0.1. Furthermore, NPR materials can have an isotropic Poisson's ratio (e.g., Poisson's ratio is the same in all directions) or an anisotropic Poisson's ratio (e.g., Poisson's ratio when the material is strained in one direction differs from Poisson's ratio when the material is strained in a different direction). In some cases, the cross-section of the artificial hair fiber 520 that includes an NPR material can have four inwardly-directed walls instead of two inwardly-directed walls. Generally, the cross section of the artificial hair fiber 520 can have any appropriate shape.

In some cases, the artificial hair fibers described in this specification can be made from a composite NPR-PPR material, e.g., the artificial hair fibers can include both regions of NPR material and regions of PPR material. Generally, NPR-PPR composite materials can be laminar composites, matrix composites (e.g., metal matrix composites, polymer matrix composites, or ceramic matrix composites), particulate reinforced composites, fiber reinforced composites, or other types of composite materials. In some examples, the NPR material is the matrix phase of the composite and the PPR material is the reinforcement phase, e.g., the particulate phase or fiber phase. In some examples, the PPR material is the matrix phase of the composite and the NPR material is the reinforcement phase.

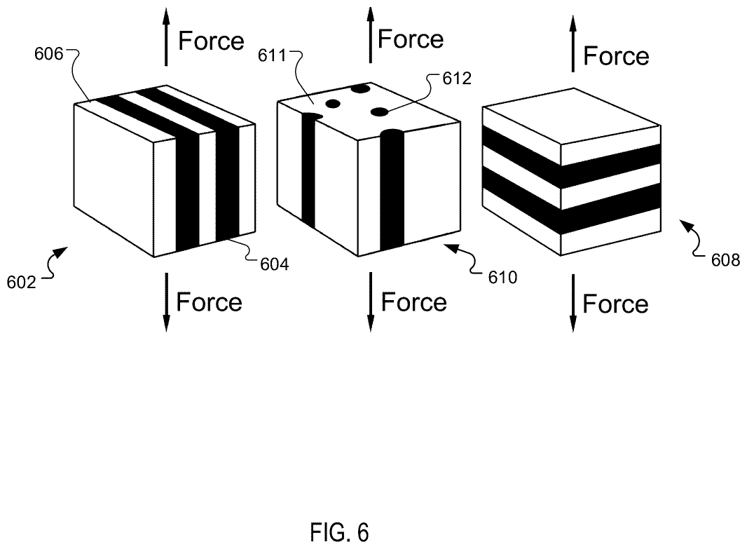

FIG. 6 illustrates examples of NPR-PPR composite materials. An NPR-PPR composite material 602 is a laminar composite including alternating layers 604 of NPR material and layers 805 of PPR material. The layers 604, 606 are arranged in parallel to a force to be exerted on the composite material 602. Although the layers 604, 606 are shown as having equal width, in some examples, a laminar composite can have layers of different widths.

An NPR-PPR composite material 608 is a laminar composite including alternating layers of NPR material and PPR material, with the layers arranged perpendicular to a force to be exerted on the material 608. In some examples, the layers of a laminar composite are arranged at an angle to the expected force that is neither perpendicular nor parallel.

An NPR-PPR composite material 612 is a matrix composite including a matrix phase 611 of NPR material with a reinforcement phase 612 of PPR material. In the material 612, the reinforcement phase 612 includes fibers of the PPR material; in some examples, the reinforcement phase 612 can include particles or other configuration. In some examples, NPR-PPR composite materials can have a matrix phase of a PPR material with a reinforcement phase of an NPR material.

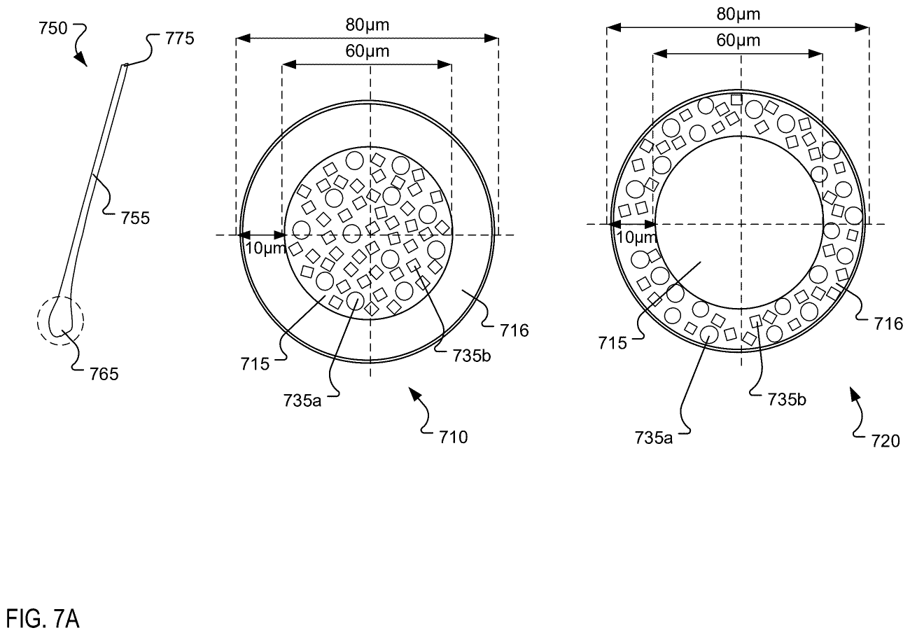

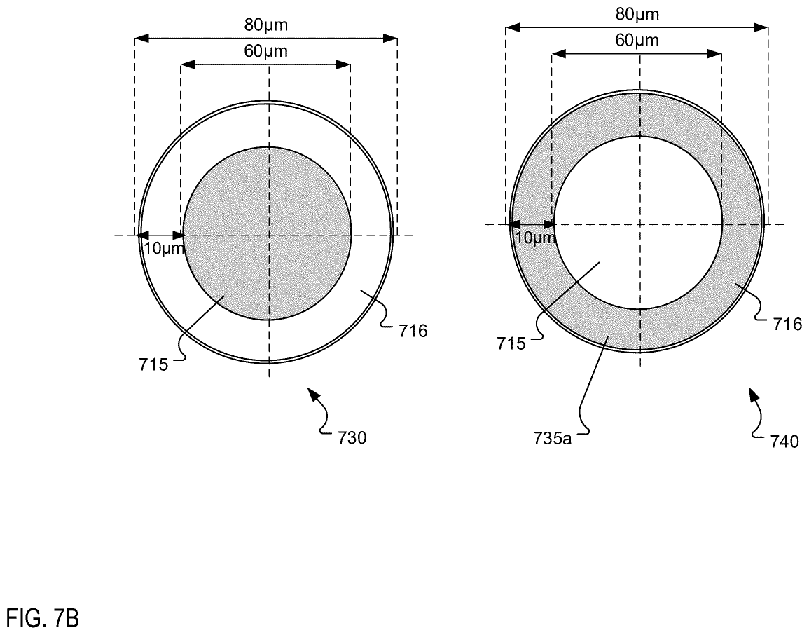

FIG. 7A illustrates an example artificial hair fiber 750 made from a composite material having both negative and positive Poisson's ratios. PPR materials of the artificial hair fiber 750 can be any appropriate PPR materials, e.g., acrylic, modacrylic, nylon, polyester, a polymer, or any other appropriate PPR material.

The artificial hair fiber 750 includes an elongated artificial hair element 755 that can generally have any appropriate length. In some cases, a body of the elongated artificial hair element 755 can have an increasing diameter from a first end to a second end. Alternatively, the body of the artificial hair element 755 can have a substantially constant diameter. The second end can have a root portion 765. The root portion 765 can have a bulb shape, a teardrop shape, or any other appropriate shape, and can generally have a wider diameter than a tip 775 of the elongated artificial hair element 755. In some cases, the root portion 765 can be configured to be implanted into human tissue. In some cases, the root portion 765 can be configured to be integrated into a hairpiece, e.g., a wig, or any other appropriate hairpiece.

In some implementations of the artificial hair fiber 750, the root portion 765 can include an NPR material. As a particular example, the root portion can include (or be made of) the NPR material, while the remaining portion of the artificial hair fiber 750 (e.g., that does not include the root portion 765) can be made of a PPR material. In some cases, the NPR material included in the root portion 765 can be a porous NPR material, e.g., a foam material. Generally, a foam is a multi-phase composite material in which one phase is gaseous and the one or more other phases are solid (e.g., polymeric, ceramic, or metallic). Foams can be closed-cell foams, in which each gaseous cell is sealed by solid material; open-cell foams, in which the each cell communicates with the outside atmosphere; or mixed, in which some cells are closed and some cells are open.

An example of an NPR foam structure is a re-entrant structure, which is a foam in which the walls of the cells are concave, e.g., protruding inwards toward the interior of the cells. In a re-entrant foam, compression applied to opposing walls of a cell will cause the four other, inwardly directed walls of the cell to buckle inward further, causing the material in cross-section to compress, such that a compression occurs in all directions. Similarly, tension applied to opposing walls of a cell will cause the four other, inwardly directed walls of the cell to unfold, causing the material in cross-section to expand, such that expansion occurs in all directions. NPR foams can have a Poisson's ratio of between −1 and 0, e.g., between −0.8 and 0, e.g., −0.8, −0.7, −0.6, −0.5, −0.4, −0.3, −0.2, or −0.1. NPR foams can have an isotropic Poisson's ratio (e.g., Poisson's ratio is the same in all directions) or an anisotropic Poisson's ratio (e.g., Poisson's ratio when the foam is strained in one direction differs from Poisson's ratio when the foam is strained in a different direction).

An NPR foam can be polydisperse (e.g., the cells of the foam are not all of the same size) and disordered (e.g., the cells of the foam are randomly arranged, as opposed to being arranged in a regular lattice). An NPR foam can have a characteristic dimension (e.g., the size of a representative cell, such as the width of the cell from one wall to the opposing wall) ranging from 0.1 μm to about 3 mm, e.g., about 0.1 μm, about 0.5 μm, about 1 μm, about 10 μm, about 50 μm, about 100 μm, about 900 μm, about 1 mm, about 2 mm, or about 3 mm.

Although the above example of a porous NPR material (e.g., NPR foam) is provided with reference to the root portion 765 of the artificial hair fiber 750, generally, any portion of the artificial hair fiber 750 can include the porous NPR material. In some cases, when the artificial heir fiber 750 that includes porous NPR material in its root portion 765 is implanted into human tissue, the tissue can grow in and around the pores of the porous NPR material thereby more firmly fixing the hair to the body and preventing the hair from falling out.

FIG. 7A also illustrates a first example 710 of a cross-section of the elongated artificial hair element 755 and a second example 720 of the cross-section of the elongated artificial hair element 755.

The cross-section of the elongated hair element 755 can generally have any appropriate diameter, e.g., less than 120 micrometers, less than 110 micrometers, less than 100 micrometers, less than 90 micrometers, less than 80 micrometers, less than 70 micrometers, or any other appropriate diameter.

As illustrated in FIG. 7A, the elongated artificial hair element 755 can include an inner portion 715 (e.g., a core) and an outer portion 716 (e.g., a coating). The inner portion 715 is illustrated as having a diameter of 60 micrometers. However, the inner portion 715 can generally have any appropriate diameter, e.g., less than 100 micrometers, less than 90 micrometers, less than 80 micrometers, less than 70 micrometers, less than 60 micrometers, less than 50 micrometers, or any other appropriate diameter.

Integration of NPR materials into the artificial hair fiber 750 can take various forms. In one example, the NPR materials can be integrated into the core portion 715 of the elongated artificial hair element 755. The example 710 in FIG. 7 shows NPR regions 735a embedded in PPR regions 735b of the inner portion 715 of the artificial elongated hair element 755. The NPR regions 735a can represent between 5% and 95% by volume of the overall inner portion 715 of the elongated artificial hair element 755, such as between 5% and 15%, between 15% and 25%, between 25% and 35%, between 35% and 45%, between 45% and 55%, between 65% and 75%, between 75% and 85%, or between 85% and 95%. The example 710 further shows the outer portion 716 that includes PPR materials. Generally, absolute and relative thicknesses, widths, and lengths of NPR regions and PPR regions can depend on, among other possible factors, a shape, size, and length of the artificial hair fiber 750, and choices of the NPR material and, if applicable, the PPR material.

The NPR regions 735a and the PPR regions 735b can take a variety of different forms. In some cases, the NPR regions 735a can be dispersed in a matrix of PPR regions 735b in the elongated artificial hair element 755. In some cases, the NPR regions 735a can be, e.g., individual particles that are dispersed in the PPR regions 735b of the elongated artificial hair element 755. In some cases, the NPR regions 735a can be, e.g., elongated fibers that at least partially extend through the length of the elongated artificial hair element 755. Generally, the NPR regions 735a and the PPR regions 735b included in the artificial hair element 755 can have any appropriate configuration.

In some implementations, the PPR material included in the outer portion 716 of the elongated artificial hair element 755 can have characteristics suitable for contact with tissue, e.g., the PPR material can be non-toxic. This can allow for the use of NPR materials in the inner portion 715 that may not meet these standards. In some cases, the NPR materials can have characteristics suitable for contact with tissue.

FIG. 7B illustrates a third example 730 of a cross-section of the elongated artificial hair element 755 and a fourth example 740 of the cross-section of the elongated artificial hair element 755. The dimensions of the cross-sections illustrated in FIG. 7B can be similar to those described above with reference to FIG. 7A. However, in the example 730, all, or substantially all, of the inner portion 715 of the artificial hair element 755 is made of NPR materials, while the outer portion 716 is made from PPR materials. In the example 740, all, or substantially all, of the outer portion 716 of the artificial hair element 755 is made of NPR materials, while the inner portion 715 is made from PPR materials. In some cases, all, or substantially all, of the inner portion 715 and the outer portion 716 of the artificial hair element 755 can be made of NPR materials.

As described above, in addition to the elongated artificial hair element 755, the artificial hair fiber 750 can further include the root portion 765. Referring to FIG. 7A, in some cases, the root portion 765 of the artificial hair fiber 750 can be made from a material, or a combination of materials, that differs from (or is the same as) the materials that are included in the elongated artificial hair element 755. For example, the root portion 765 can include NPR materials in a different (or the same) configuration as the elongated artificial hair element 755. In some cases, the root portion 765 can include a composite material having both negative and positive Poisson's ratios, e.g., in a similar configuration as described above with reference to examples 710 and 720 and illustrated in FIG. 7A. For example, the root portion 765 can similarly include a core portion and an inner portion (not shown), either (or both) of which can include NPR regions embedded in PPR regions. In some implementations, all, or substantially all, of the core portion, the inner portion, or both, of the root portion 765 of the artificial hair fiber 750 can be made from NPR materials, in a similar configuration as described above with reference to examples 730 and 740 and illustrated in FIG. 7B. The NPR regions and/or the PPR regions in the root portion 765 of the artificial hair fiber 750 can have any appropriate form, e.g., can be filaments extending through the root portion 765, individual particles, the NPR regions can be dispersed through the PPR regions. Generally, the NPR regions and the PPR regions in the root portion 765 of the artificial hair fiber 750 can be configured in any appropriate manner.

Integrating NPR materials into the root portion 765 of the artificial hair fiber 750 can have many advantages. For example, as described above, NPR materials expand in transverse direction in response to expansion in longitudinal direction, e.g., when stretched. Accordingly, when artificial hair fibers that include NPR materials in the root portion 765 are implanted into human tissue, or integrated into hairpieces such as wigs, the strength of the artificial hair fibers at the root portion 765 can be increased, thereby more securely attaching them to human tissue or hair pieces.

NPR material included in artificial hair fibers, according to implementations of this disclosure, need not be entirely contained within a PPR material. In some cases, NPR material can be exposed on an outer surface of the artificial hair fiber 750. For example, as illustrated in the example 720 in FIG. 7A, and the example 740 in FIG. 7B, the outer portion 716 of the artificial hair fiber 750 can include NPR regions 735a some of which may be exposed to the outer surface of the artificial hair fiber 750. In various implementations, NPR material included in the artificial hair fiber 750 may be entirely embedded in a PPR material 735b, partially embedded in a PPR material 735b and partially exposed, or entirely exposed, e.g., for the artificial hair fiber 750 or a component of the artificial hair fiber 750 made entirely of an NPR material 735a (also within the scope of this disclosure), or, for the artificial hair fiber 750 in which an NPR material 735a forms the outer portion 740.

Having an NPR material form, or at least partially form, an outer portion 740 of the artificial hair fiber 750 can provide advantages in some implementations. The tendency of the NPR material to compress in a transverse direction, rather than expand, in response to compression can help to maintain homeostasis when the artificial hair fiber 750 is implanted into human tissue. The outer NPR material may compress in response to outward-directed force from the internal PPR material, and/or may expand in response to an inward-directed force (a pulling force) from the internal PPR material, maintaining a more constant total volume of the artificial hair fiber 750 over time. Moreover, the NPR material may be lighter and may have improved stress/strain characteristics when compared to the PPR material.

NPR portions 735a of the artificial hair fiber 750 can be produced in a variety of ways. In some implementations, an initial PPR material (sometimes referred to as a “precursor material”) is converted into the NPR material. For example, a porous PPR sponge or foam can be transformed to change its structure into a structure that exhibits a negative Poisson's ratio. In some examples, NPR foams are produced by transformation of nanostructured or microstructured PPR materials, such as nanospheres, microspheres, nanotubes, microtubes, or other nano- or micro-structured materials, into a foam structure that exhibits a negative Poisson's ratio. The transformation of a PPR foam or a nanostructured or microstructured material into an NPR foam can involve thermal treatment (e.g., heating, cooling, or both), application of pressure, or a combination thereof. In some examples, PPR materials, such as PPR foams or nanostructured or microstructured PPR materials, are transformed into NPR materials by chemical processes, e.g., by using glue. In some examples, NPR materials are fabricated by additive manufacturing, e.g., three-dimensional (3D) printing techniques, or other appropriate additive manufacturing technique.

Other methods can also be used to fabricate an artificial hair fiber 750 including an NPR material or an NPR-PPR composite material. For example, various additive manufacturing (e.g., 3D printing) techniques, or other appropriate additive manufacturing technique, can be implemented. In some examples, different components of the artificial hair fiber 750 are made by different techniques. For example, an internal NPR portion may be 3D printed while the outer PPR portion is not, or vice versa.

Therefore, in accordance with the implementations of this disclosure, artificial hair fibers that include NPR materials are described.

Various modifications will be apparent from the foregoing detailed description. For example, structures and processes described in associated with one type of medical implant (e.g., a bone plate, a hip prosthesis component, a finger joint prosthesis component, or a dental implant) may be equally applicable for other types of medical implants, including other types of medical prostheses. Further, features described above in connection with different implementations may, in some cases, be combined in the same implementation. In some instances, the order of the process steps may differ from that described in the particular examples above.

Accordingly, other implementations are also within the scope of the claims.

What is claimed is:

1. An artificial hair, comprising:
an elongated artificial hair element comprising a negative Poisson's ratio (NPR) material having a Poisson's ratio of between 0 and −1.

2. The artificial hair of claim 1, wherein the elongated artificial hair element is configured to be implanted into human tissue.

3. The artificial hair of claim 1, wherein the elongated artificial hair element further comprises a positive Poisson's ratio (PPR) material.

4. The artificial hair of claim 1, further comprising:
a coating covering at least a portion of an outer surface of the elongated artificial hair element, wherein the coating comprises the negative Poisson's ratio (NPR) material.

5. The artificial hair of claim 4, wherein the coating comprises one or more elongated fibers of the negative Poisson's ratio (NPR) material that at least partially extend through the elongated artificial hair element.

6. The artificial hair of claim 4, wherein the coating comprises a plurality of particles of the negative Poisson's ratio (NPR) material.

7. The artificial hair of claim 4, wherein the coating comprises a matrix of the negative Poisson's ratio (NPR) material and a positive Poisson's ratio (PPR) material.

8. The artificial hair of claim 1, wherein the elongated artificial hair element comprises a body having an increasing diameter from a first end to a second end, the second end comprising a root portion that is configured to be implanted into human tissue.

9. The artificial hair of claim 8, wherein the root portion comprises the negative Poisson's ratio (NPR) material having a Poisson's ratio of between 0 and −1.

10. The artificial hair of claim 9, wherein a portion of the elongated artificial hair element other than the root portion comprises a positive Poisson's ratio (PPR) material.

11. The artificial hair of claim 9, wherein the root portion comprises:
one or more elongated fibers of the negative Poisson's ratio (NPR) material that at least partially extend through the root portion, or a plurality of particles of the negative Poisson's ratio (NPR) material.

12. The artificial hair of claim 9, wherein the negative Poisson's ratio (NPR) material is a porous negative Poisson's ratio (NPR) material.

13. The artificial hair of claim 9, wherein the root portion comprises a matrix of the negative Poisson's ratio (NPR) material and a positive Poisson's ratio (PPR) material.

14. The artificial hair of claim 1, wherein the elongated artificial hair element comprises a core portion that extends through the elongated artificial hair element, and wherein the core portion comprises the negative Poisson's ratio (NPR) material.

15. The artificial hair of claim 14, wherein the core portion comprises one or more elongated fibers of the negative Poisson's ratio (NPR) material that at least partially extend through the elongated artificial hair element.

16. The artificial hair of claim 14, wherein the core portion comprises a plurality of particles of the negative Poisson's ratio (NPR) material.

17. The artificial hair of claim 14, wherein the core portion comprises a matrix of the negative Poisson's ratio (NPR) material and a positive Poisson's ratio (PPR) material.

18. A method for making an artificial hair, the method comprising:
forming an elongated artificial hair element from a negative Poisson's ratio (NPR) material having a Poisson's ratio of between 0 and −1.

19. The method of claim 18, wherein forming the elongated artificial hair element comprises forming the elongated artificial hair element using an additive manufacturing technique.

20. The method of claim 18, wherein forming the elongated artificial hair element comprises:
forming the elongated artificial hair element from a matrix of the negative Poisson's ratio (NPR) material and a positive Poisson's ratio material (PPR).
